(19) United States
(12) Patent Application Publication
(10) Pub. No.: US 2023/0012417 A1
(43) Pub. Date: Jan. 12, 2023

(54) LEADLESS PACING DEVICE WITH CORONARY SINUS LEADLET

(71) Applicant: Medtronic, Inc., Minneapolis, MN (US)

(72) Inventors: Kaileigh E. Rock, Mahtomedi, MN (US); Kathryn E. Hilpisch, Cottage Grove, MN (US); Douglas S. Hine, Forest Lake, MN (US); Matthew D. Bonner, Plymouth, MN (US)

(21) Appl. No.: 17/808,424

(22) Filed: Jun. 23, 2022

Related U.S. Application Data
(60) Provisional application No. 63/219,309, filed on Jul. 7, 2021.

(51) Int. Cl. A61N 1/05; A61N 1/362; A61N 1/372; A61N 1/375

(52) U.S. Cl. CPC A61N 1/05; A61N 1/362; A61N 1/37205; A61N 1/37518

(57) ABSTRACT

A medical system includes an implantable medical device configured to be positioned within an atrium of a heart. The implantable medical device includes a housing carrying a return electrode, a first leadlet, a second leadlet, and a fixation device. The medical system may be configured to deliver a variety of therapies, including one or more of ventricle-from-atrium cardiac therapy (“VfA therapy”), left bundle branch pacing therapy (“LBB therapy”), or cardiac resynchronization therapy (“CRT”).

This application claims the benefit of U.S. Provisional patent application Ser. No. 63/219,309 (filed Jul. 7, 2021), which is entitled, “LEADLESS PACING DEVICE WITH CORONARY SINUS LEADLET” and is incorporated by reference herein in its entirety.

### TECHNICAL FIELD

This disclosure is related to implantable medical systems.

### BACKGROUND

Various types of implantable medical leads have been implanted for treating or monitoring one or more conditions of a patient. Such implantable medical leads may be adapted to allow medical devices to monitor or treat conditions or functions relating to heart, muscle, nerve, brain, stomach endocrine organs or other organs and their related functions. Implantable medical leads include electrodes and/or other elements for physiological sensing and/or therapy delivery. Implantable medical leads allow the sensing/therapy elements to be positioned at one or more target locations for those functions, while the medical devices electrically coupled to those elements via the leads are at different locations.

Implantable medical leads, e.g., distal portions of elongate implantable medical leads, may be implanted at target locations selected to detect a physiological condition of the patient and/or deliver one or more therapies. For example, implantable medical leads may be delivered to locations within an atria or ventricle to sense intrinsic cardiac signals and deliver pacing or antitachyarrhythmia shock therapy from a medical device coupled to the lead. In other examples, implantable medical leads may be tunneled to locations adjacent a spinal cord or other nerves for delivering pain therapy from a medical device coupled to the lead. Implantable medical leads may include anchoring components to secure a distal end of the lead at the target location.

Some implantable medical systems, such as cardiac pacemakers, are sized to be completely implanted within one of the chambers of the heart, and may include electrodes integrated with or attached to the device housing rather than leads. Such implantable medical systems may include fixation components to secure the pacemaker to cardiac tissue at a target location. Some implantable medical systems provide dual chamber functionality, by sensing and/or stimulating the activity of both atria and ventricles, or other multi-chamber functionality. A implantable medical systems may provide multi-chamber functionality via leads that extend to respective heart chambers, or multiple implantable medical systems may provide multi-chamber functionality by being implanted in respective chambers.

### SUMMARY

In an example, a medical system comprises: an implantable medical device configured to be positioned within an atrium of a heart and comprising a housing carrying a return electrode, wherein the housing comprises a proximal end and a distal end; a first leadlet comprising a first proximal end and a first distal end, wherein the first proximal end is attached to the implantable medical device, and wherein the first leadlet carries a first electrode; and a fixation device attached to the distal end of the housing, wherein the first leadlet is configured to extend within a coronary sinus of the heart when the fixation device secures the distal end to heart tissue in the atrium.

In an example, a method of implanting a medical system comprises: positioning an implantable medical device within an atrium of a heart, wherein the implantable medical device comprises a housing carrying a return electrode, wherein the housing comprises a proximal end and a distal end; securing a fixation device, attached to the distal end of the housing, to the heart tissue; and extending a first leadlet into a coronary sinus of the heart, wherein the first leadlet comprises a first proximal end and a first distal end, wherein the first proximal end is attached to the implantable medical device, and wherein the first leadlet carries a first electrode.

In an example, a medical system comprises: an implantable medical device configured to be positioned within an atrium of a heart and comprising a housing carrying a return electrode, wherein the housing comprises a proximal end and a distal end; a first leadlet comprising a first proximal end and a first distal end, wherein the first proximal end is attached to the implantable medical device, and wherein the first leadlet carries a first electrode; a second leadlet comprising a second proximal end and a second distal end, wherein the second leadlet carries a second electrode, wherein the second proximal end is attached to the implantable medical device, and wherein the second distal end is configured to penetrate the heart tissue when a distal force is applied to the second leadlet to form a puncture sized to allow the second electrode to be inserted therethrough; a third electrode disposed on the distal end of the housing; and a fixation device attached to the distal end of the housing, wherein the first leadlet is configured to extend within a coronary sinus of the heart when the fixation device secures the distal end to heart tissue in the atrium, and wherein the third electrode is configured to contact a surface of the heart tissue when the fixation device secures the implantable medical device to the heart tissue.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example implantable medical system with a first leadlet extending into a coronary sinus of a heart.

FIG. 2A is a conceptual diagram illustrating an example implantable medical device and an elongate member.

FIG. 2B is a conceptual diagram illustrating an example implantable medical device including a fixation device.

FIG. 3 is a block diagram illustrating circuitry of an example implantable medical device.

FIG. 4 is a conceptual diagram illustrating an example medical system including an implantable medical device and a guidewire.

FIG. 5 is a flow diagram of an example technique for positioning a medical system within a heart.

### DETAILED DESCRIPTION

This disclosure describes a medical system configured to position one or more electrodes within a heart. The medical system includes an implantable medical device (“IMD”) configured to be positioned within an atrium of the heart. The IMD may house electric circuitry within an enclosure that are electrically coupled to one or more electrodes of the IMD. The electrodes may be configured to sense electrical signals from and/or deliver therapy signals to the heart.

The IMD may include a first leadlet and, in some examples, may include a second leadlet. The first leadlet may be configured to extend within a coronary sinus of the heart when the IMD is attached to tissues of the heart. The first leadlet may define a first proximal end and a first distal end. The first proximal end may be attached to a housing of the IMD. In examples, the first leadlet is configured as a substantially free-floating lead within the coronary sinus, such that the first distal end is a free end (e.g., unattached to tissue) within the coronary sinus. In some examples, the first distal end may be configured to penetrate coronary sinus tissue when the first leadlet extends into the coronary sinus, such that the first leadlet is substantially anchored within the coronary sinus. The first distal end may be configured to penetrate the tissue when a distal force is applied to the first leadlet.

A first leadlet body between the first proximal end and the first distal end may be configured to be positioned within the coronary sinus when a fixation device of the IMD secures the IMD to heart tissue (e.g., in the atrium). The first leadlet may carry (e.g., mechanically support) a first electrode. The first electrode may be configured to deliver therapy to the heart. In some examples, the first electrode may be a multi-polar electrode (including two electrodes, four electrodes, etc.) configured to deliver left ventricular therapy (“LV therapy”) in a substantially non-invasive manner when at least a portion of the first leadlet is positioned within the coronary sinus. In such examples, the electrodes of the multi-polar electrode may be configured to independently deliver electrical stimulation. For example, a first electrode may be electrically connected to the electric circuitry via a first conductor and a second electrode may be electrically connected to the electric circuitry via a second conductor insulated from the first conductor, such that the first electrode and the second electrode may independently deliver signals to and/or sense signals from a heart of a patient.

In some examples, the first leadlet may be configured to cause the first electrode to insert into tissue of the coronary sinus. The first electrode may be a penetrating electrode configured to penetrate the tissue. For example, the first distal end may be configured to form a puncture sized to allow the first electrode to be inserted therethrough when a distal force is applied to the first leadlet. The first leadlet may be configured to receive an elongate member (e.g., a long and narrow, but not necessarily stretched, member), such as a stylet, configured to exert or cause the first leadlet to exert the distal force on the first distal end. In some examples, the first leadlet may define a first bearing structure (e.g., a lumen, a casing, etc.) configured to transmit a distal force to the first distal end when the elongate member exerts the distal force on the first bearing structure.

In some cases, the IMD includes a second electrode positioned proximate to a distal end of the IMD. The second electrode may be a deep (e.g., rigid) electrode configured to penetrate tissue. The second electrode may optionally be carried by a second leadlet defining a second proximal end and a second distal end. For example, the second leadlet may mechanically support the second electrode near the second distal end. The second proximal end may be attached to the housing of the IMD. The second distal end may be configured to penetrate tissue of the heart when a distal force is applied to the second leadlet (e.g., via IMD) to form a puncture sized to allow the second electrode to be inserted therethrough. The second distal end may be configured to be positioned within heart tissue when the fixation device secures the IMD to heart tissue (e.g., in the atrium).

The second electrode may be configured to deliver therapy to the heart. Like the first electrode, the second electrode may be a multi-polar electrode (including two electrodes, four electrodes, etc.), where the electrodes of the multi-polar electrode may be configured to independently deliver electrical stimulation. The second electrode may be configured to deliver ventricle-from-atrium cardiac therapy (“VfA therapy”) when the second distal end is implanted in a right atrium of the heart and penetrates into the ventricular tissue. Additionally or alternatively, the second electrode may be configured to deliver left bundle branch pacing therapy (“LBB therapy”) when the second distal end is implanted in a right ventricle septum of the heart.

The second leadlet may be configured to insert or cause insertion of the second electrode into heart tissue. In some examples, the second leadlet may be configured to receive an elongate member, such as a stylet, configured to exert a distal force on the second distal end. In some examples, the second leadlet may define a second bearing structure (e.g., a lumen, a casing, etc.) configured to transmit a distal force to the second distal end when the elongate member exerts the distal force on the second bearing structure.

In some examples, the medical system (e.g., the IMD) may carry a third electrode configured to contact a surface of heart tissue when the fixation device secures the IMD to heart tissue in the atrium. The third electrode may be disposed (e.g., attached) on the housing of the IMD, such as the distal end of the housing. Alternatively, a third leadlet defining a third proximal end and a third distal end may mechanically support the third electrode, for example, near the third distal end. The third electrode may be configured to deliver therapy to the heart. In examples, the third electrode is an atrial electrode. The third electrode may be configured to provide stimulation to the heart.

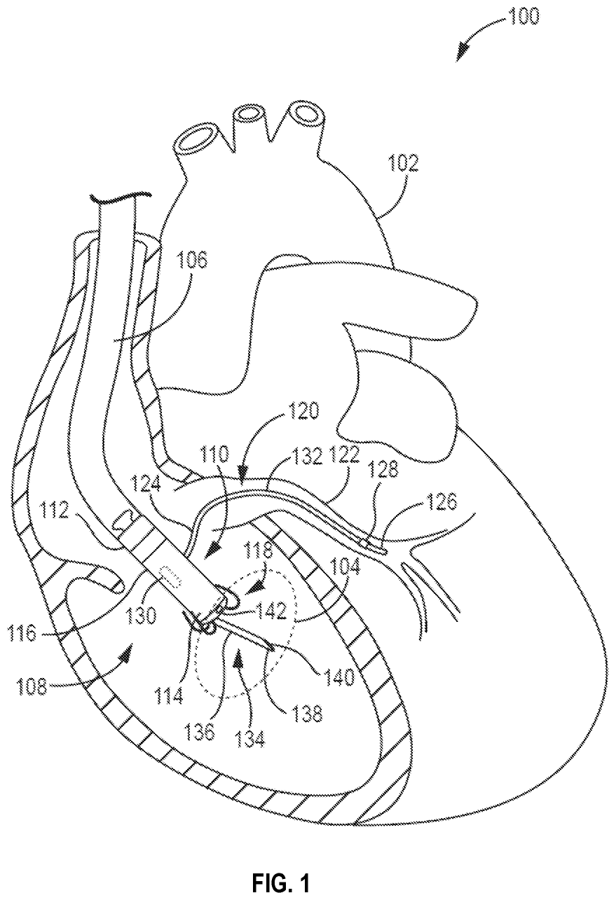

FIG. 1 is a conceptual diagram illustrating a portion of an example medical device system 100 configured to be positioned at an implant site 104 within a heart 102. The implant site 104 may include an appendage or triangle of Koch region of a right atrium (RA) of a heart 102 of a patient. In some examples, implant site 104 may include other portions of heart 102, such as in the right ventricle (RV) of a heart 102 of a patient, or other locations within a body of the patient.

Medical device system 100 may include a delivery catheter 106 configured to deliver an implantable medical device 108 (“IMD 108”) to implant site 104. Delivery catheter 106 may define a lumen (“catheter lumen”) configured to allow IMD 108 to translate through the catheter lumen. Delivery catheter 106 may define a lumen opening to the catheter lumen at a distal end of delivery catheter 106. The catheter lumen and the lumen opening may be sized to allow IMD 108 to translate through the catheter lumen and the lumen opening to position IMD 108 within or in the vicinity of implant site 104.

Delivery catheter 106 may be configured to allow a clinician to deliver medical system 100 through a vena cava (e.g., the superior vena cava (SVC) or inferior vena cava (IVC)) for implantation of IMD 108 within an atrium (e.g., the RA) of heart 102. For example, delivery catheter 106 may be intravenously transited through the vena cava such that a distal section of delivery catheter 106 passes into the RA. As such, delivery catheter 106 may be configured to allow the clinician to position the catheter distal end in proximity to implant site 104 as the catheter distal section transitions from the vena cava (e.g., the SVC or IVC) into the atrium (e.g., RA). Implant site 104 may include a portion of a right ventricle (RV), an appendage or triangle of Koch region of the RA, or some other portion of heart 102, or other locations within a body of a patient. Other pathways or techniques may be used to guide delivery catheter 106 into other implant sites within the body of the patient.

IMD 108 may include a housing 110 defining a proximal end 112 of IMD (“IMD proximal end 112”) and a distal end 114 of IMD 108 (“IMD distal end 114”). Housing 110 may include a housing wall 116 extending between IMD proximal end 112 and IMD distal end 114. A fixation device 118 configured to engage tissue may be attached to IMD distal end 114. Fixation device 118 may include, for example, one or more elongate tines, such as fixation tines, configured to substantially maintain an orientation of IMD 108 with respect to implant site 104. Fixation device 118 may include fixation tines of any shape, including helically-shaped fixation tines. Fixation device 118 may be configured to penetrate tissue in the vicinity of implant site 104 to substantially maintain an orientation of IMD 108. For example, fixation device 118 may be configured to affix IMD 108 to implant site 104 by substantially affixing IMD distal end 114 to the tissue. In examples, fixation device 118 is attached to IMD distal end 114.

IMD 108 may include a first leadlet 120 and, in some examples, may include a second leadlet 134. First leadlet 120 may be configured to extend within a coronary sinus 122 of heart 102. First leadlet 120 may define a first proximal end 124 and a first distal end 126. First proximal end 124 may be attached to IMD 108, such as to IMD proximal end 112, IMD distal end 114, housing wall 116, or another portion of IMD 108. In some examples, first distal end 126 may be configured to penetrate coronary sinus tissue when a distal and/or rotational force is applied to first leadlet 120.

A first leadlet body 132 between first proximal end 124 and first distal end 126 may be configured to be positioned within coronary sinus 122 when fixation device 118 secures IMD distal end 114 to heart tissue (e.g., in the atrium). First leadlet 120 may carry (e.g., mechanically support) a first electrode 128. For example, first leadlet 120 may carry first electrode 128 on first leadlet body 132, first distal end 126, or some other portion of first leadlet 120. First electrode 128 may be configured to deliver therapy to heart 102. In some examples, first electrode 128 may be a multi-polar electrode (including two electrodes, four electrodes, etc.) configured to deliver LV therapy in a substantially non-invasive manner when at least a portion of first leadlet 120 is positioned within coronary sinus 122. In such examples, the electrodes of the multi-polar electrode may be configured to independently deliver electrical stimulation. First electrode 128 may be electrically coupled to electric circuitry 130 (“circuitry 130”) housed within IMD 108. Circuitry 130 may be one or more of sensing circuitry (e.g., for sensing cardiac signals), therapy delivery circuitry (e.g., for generating cardiac pacing pulses), or processing circuitry for controlling the functionality of IMD 108. Circuitry 130 may be mechanically supported within housing 110 and be configured to deliver LV therapy to heart 102 using first electrode 128 and a return electrode carried by housing 110.

In some examples, first leadlet 120 may be configured to cause first electrode 128 to insert into tissue of coronary sinus 122. First electrode 128 may be a penetrating electrode configured to penetrate the tissue. For example, first distal end 126 may be configured to form a puncture sized to allow first electrode 128 to be inserted therethrough when a distal force is applied to first leadlet 120. First leadlet 120 may be configured to receive an elongate member, such as a stylet, configured to exert or cause first leadlet 120 to exert the distal force on first distal end 126. In some examples, first leadlet 120 may define a first bearing structure (e.g., a lumen, a casing, etc.) configured to transmit a distal force to first distal end 126 when the elongate member exerts the distal force on the first bearing structure. For example, the first bearing structure may be an inner lumen with a surface configured to receive a distal force. The first bearing structure may be configured to receive an elongate member, and the elongate member may be configured to mechanically communicate with the surface of the first bearing structure, in this way exerting a distal force on the first bearing structure and, thus, first distal end 126.

In some examples, first distal end 126 may include a fixation structure (e.g., one or more barbs) configured to resist motion of first electrode 128 in a proximal direction and/or distal direction when first distal end 126 is implanted in tissue of coronary sinus 122. For example, the fixation structure may be configured to resist a translation of first distal end 126 in a proximal direction when first distal end 126 is implanted in tissue of coronary sinus 122. In examples, the fixation structure is configured such that, when first distal end 126 is implanted within tissue, the fixation structure causes first leadlet 120 to exert a first reaction force when a distally directed force having a first magnitude is exerted on first distal end 126, and exert a second reaction force when a proximally directed force having the first magnitude is exerted on first distal end 126. In such examples, the magnitude of the first reaction force may be less than or equal to the magnitude of the distally directed force (allowing movement of first leadlet 120 in a distal direction), and the magnitude of the second reaction force may be equal to or greater than the magnitude of the proximally directed force (resisting movement of first leadlet 120 in a proximal direction). Hence, when first distal end 126 is implanted within tissue and fixation device 118 attaches to tissues of heart 102, first leadlet 120 may be substantially anchored at first proximal end 124 and first distal end 126.

In some examples, first leadlet 120 may be configured to position first electrode 128 within coronary sinus 122 (e.g., as a non-penetrating electrode). First leadlet 120 may carry (e.g., mechanically support) first electrode 128 on some portion of leadlet body 132. IMD 108 may be configured such that the portion of leadlet body 132 carrying first electrode 128 may position within coronary sinus 122 when fixation device 118 attaches IMD 108 to the tissue in the heart, such that first leadlet 120 establishes first electrode 128 as a free-floating electrode within coronary sinus 122.

In some examples, IMD 108 includes second electrode 140 positioned proximate to IMD distal end 114. Second electrode 140 may be configured to penetrate tissue of heart 102. In some examples, second electrode 140 may be carried by a second leadlet 134 defining a second proximal end 136 and a second distal end 138. For example, second leadlet 134 may mechanically support second electrode 140 near second distal end 138. Second proximal end 136 may be attached to IMD 108, such as to IMD distal end 114 or another portion of IMD 108. Second distal end 126 may be configured to penetrate tissue of heart 102 when a distal force is applied to second leadlet 134 (e.g., via IMD 108) to form a puncture sized to allow second electrode 140 to be inserted therethrough. Second distal end 138 may be configured to be positioned within heart tissue when fixation device 118 secures IMD distal end 114 to heart tissue (e.g., in the atrium). In examples, second electrode 140 is configured to penetrate the heart tissue,

Second electrode 140 may be configured to deliver therapy to heart 102. Second electrode 140 may be a multi-polar electrode (including two electrodes, four electrodes, etc.), where the electrodes of the multi-polar electrode may be configured to independently deliver electrical stimulation. Second electrode 140 may be configured to deliver VfA therapy when second distal end 138 penetrates a right atrium of heart 102 and reaches ventricular tissue. Additionally or alternatively, second electrode 140 may be configured to deliver LBB therapy when second distal end 138 is implanted in a right ventricle septum of heart 102. Second electrode 140 may be electrically coupled to circuitry 130 housed within IMD 108. Circuitry 130 may be configured to deliver VfA therapy and/or LBB therapy to heart 102 using second electrode 140 and a return electrode carried by housing 110.

As described above, second leadlet 134 may be configured to insert or cause insertion of second electrode 140 into heart tissue. In some examples, second leadlet 134 may be configured to receive an elongate member, such as a stylet, configured to exert a distal force on second distal end 138. In some examples, second leadlet 134 may define a second bearing structure (e.g., a lumen, a casing, etc.) configured to transmit a distal force to second distal end 138 when the elongate member exerts the distal force on the second bearing structure. For example, the second bearing structure may be a casing extending from an outer surface of second leadlet 134 configured to receive a distal force. The second bearing structure may be configured to receive an elongate member, and the elongate member may be configured to mechanically communicate with the second bearing structure, in this way exerting a distal force on the second bearing structure and, thus, second distal end 138. In examples, second leadlet 134 may have a sufficient stiffness to cause insertion of second electrode 140 when IMD 108 imparts a distal force on second leadlet 134 (e.g., on second proximal end 136).

Like first distal end 126, second distal end 138 may include a fixation structure (e.g., barbs) configured to resist motion of second electrode 140 in a proximal direction and/or distal direction when second distal end 138 is implanted in tissue of heart 102. For example, the fixation structure may be configured to resist a translation of second distal end 138 in a proximal direction when second distal end 138 is implanted in tissue of heart 102. In examples, the fixation structure is configured such that, when second distal end 138 is implanted within tissue, the fixation structure causes second distal end 138 to exert a first reaction force when a distally directed force having a first magnitude is exerted on second distal end 138, and exert a second reaction force when a proximally directed force having the first magnitude is exerted on second distal end 138. In such examples, the magnitude of the first reaction force may be less than or equal to the magnitude of the distally directed force (allowing movement of second leadlet 134 in a distal direction), and the magnitude of the second reaction force may be equal to or greater than the magnitude of the proximally directed force (resisting movement of second leadlet 134 in a proximal direction). Hence, when second distal end 138 is implanted within tissue and fixation device 118 attaches to tissues of heart 102, second leadlet 134 may be substantially anchored at second proximal end 136 and second distal end 138.

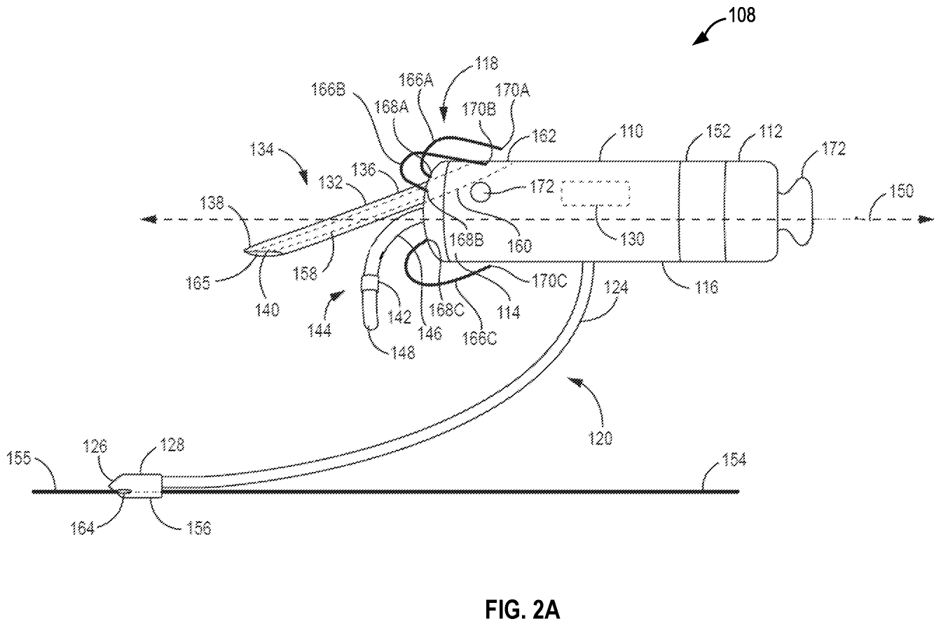

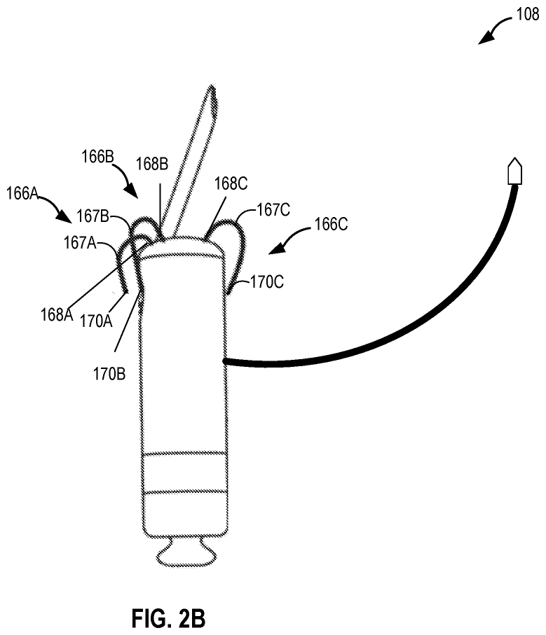

In some examples, IMD 108 may carry a third electrode 142 on IMD distal end 114. Third electrode 142 may be configured to contact a surface of heart tissue when fixation device 118 secures IMD distal end 114 to heart tissue in the atrium. In examples, third electrode 142 is a button electrode mechanically supported by (e.g., attached to) IMD distal end 114, housing wall 116, or another portion of IMD 108. Alternatively, a third leadlet 144 (FIG. 2A) defining a third proximal end 146 (FIG. 2A) and a third distal end 148 (FIG. 2A) may mechanically support third electrode 142, for example, near third distal end 148. In examples, third proximal end 146 is mechanically supported by (e.g., attached to) IMD distal end 114, housing wall 116, or another portion of IMD 108.

Third electrode 142 may be configured to deliver therapy to heart 102. In examples, third electrode 142 is an atrial electrode. Third electrode 142 may be configured to provide stimulation to heart 102. Third electrode 142 may be electrically coupled to circuitry 130 housed within IMD 108. Circuitry 130 may be configured to deliver therapy to heart 102 using third electrode 142 and a return electrode carried by housing 110.

IMD 108 may include first electrode 128, second electrode 140, and third electrode 142 in various combinations. For example, IMD 108 may include first leadlet 120 carrying first electrode 128. In another example, IMD 108 may include first leadlet 120 carrying first electrode 128 and second leadlet 134 carrying second electrode 140. In yet another example IMD 108 may include first leadlet 120 carrying first electrode 128 and third leadlet 144 carrying third electrode 142. In yet another example, IMD 108 may include first leadlet 120 carrying first electrode 128, second leadlet 134 carrying second electrode 140, and third leadlet 144 carrying third electrode 142.

FIG. 2A is a conceptual diagram illustrating IMD 108 including first leadlet 120, second leadlet 134, and third leadlet 144. IMD 108 includes housing 110 extending along longitudinal axis 150 from IMD proximal end 112 to IMD distal end 114. Housing 110 may be formed from a biocompatible and biostable metal such as titanium. In some examples, housing 110 may include a hermetically sealed housing. Housing 110 may mechanically support circuitry 130 of IMD 108 within an enclosure of housing 110. In some examples, housing 110 may mechanically support a return electrode 152 that is operably coupled (e.g., in electrical communication with) circuitry 130 of IMD 108. Housing 110 may include a nonconductive coating and define return electrode 152 as an uncoated portion of housing 110.

At least a portion of IMD 108 (e.g., housing 110) is configured to position within heart 102 (FIG. 1). In examples, IMD 108 is configured to position within an atrium (e.g., the RA) of heart 102. IMD 108 may include any suitable dimensions sufficient to allow IMD 108 to be positioned within heart 102 (FIG. 1). In some examples, an outer diameter of IMD 108 (e.g., an outer diameter of housing 110) may be between about 10 French (Fr) and about 23 Fr, such as about 20 Fr, although other outer diameters of IMD 108 are contemplated.

As shown in FIG. 2A, IMD 108 may include first leadlet 120 carrying first electrode 128, second leadlet 134 carrying second electrode 140, and third leadlet 144 carrying third electrode 142. First electrode 128, second electrode 140, and third electrode 142 may be electrically coupled to circuitry 130 and configured to receive therapy signals from IMD 108 for delivery to tissues of heart 102. In this way, circuitry 130 may be configured to delivery therapy to heart 102 using first electrode 128, second electrode 140, third electrode 142, and/or return electrode 152.

First electrode 128 and/or third electrode 142 may be configured for sensing and delivery of therapy signals to tissue in a substantially non-invasive manner. For example, first electrode 128 may be configured to provide stimulation to surface tissue of coronary sinus 122 when fixation device 118 secures IMD distal end 114 to heart 102, and second electrode 142 may be configured to provide stimulation to surface tissue in a chamber of heart 102 when fixation device 118 secures IMD distal end 114 to heart 102. In some examples, first electrode 128 and/or second electrode 140 may be configured to penetrate tissue for sensing electrical signals and/or delivery of therapy signals. First electrode 128 and second electrode 140 may each be a multi-polar electrode.

First leadlet 120 (e.g., some portion of first leadlet body 119) may be configured to extend within coronary sinus 122 of heart 102 when IMD 108 positions within heart 102. In examples, first leadlet 120 is configured to position in coronary sinus 122 as a substantially free-floating member, such that first distal end 126 is a free end unattached to tissue in coronary sinus 122. In some examples, first leadlet 120 is configured to substantially anchor in coronary sinus 122. For example, first distal end 126 may be configured to penetrate tissue in coronary sinus 122 to anchor first leadlet 120 in coronary sinus 122.

First leadlet 120 may be configured to receive an elongate member 154 (e.g., a stylet) to position first leadlet 120 within coronary sinus 122 and/or cause penetration of first distal end 126 into tissues. First leadlet 120 may be configured to receive a distal force from elongate member 154 to exert or cause first leadlet 120 to exert a distal force on first distal end 126. In some examples, first leadlet 120 defines a first bearing structure 156 (e.g., a lumen, a casing, etc.) configured to transmit the distal force to first distal end 126 when elongate member 154 exerts the distal force on first bearing structure 156. For example, as shown in FIG. 2A, first bearing structure 156 may be a casing extending from an outer surface of first leadlet 120 configured to receive a distal force. First bearing structure 156 may be configured to receive elongate member 154, and elongate member 154 may be configured to mechanically communicate with first bearing structure 156, in this way exerting a distal force on first bearing structure 156 and, thus, first distal end 126. In examples, first leadlet 120 may have a sufficient stiffness to cause insertion of first electrode 128 when IMD 108 imparts a distal force on first leadlet 120 (e.g., on first proximal end 124).

Similarly, second leadlet 134 may be configured to receive elongate member 154. Elongate member 154 may be configured to exert a distal force on second distal end 138. In some examples, second leadlet 134 may define a second bearing structure 158 (e.g., a lumen, a casing, etc.) configured to transmit a distal force to second distal end 138 when elongate member 154 exerts the distal force on second bearing structure 158. For example, as shown in FIG. 2A, second bearing structure 158 may be a lumen with a surface configured to receive a distal force. Second bearing structure 158 may be configured to receive elongate member 154, and elongate member 154 may be configured to mechanically communicate with second bearing structure 158, in this way exerting a distal force on second bearing structure 158 and, thus, second distal end 138. In examples, second leadlet 134 may have a sufficient stiffness to cause insertion of second electrode 140 when IMD 108 imparts a distal force on second leadlet 134 (e.g., on second proximal end 136).

In some examples, IMD 108 may define an inner lumen 160 configured to receive elongate member 154. Inner lumen 160 may extend from a lumen opening 162 defined by housing wall 116 toward first distal end 126 and/or second distal end 138. Elongate member 154 may be configured to extend through inner lumen 160 and toward first distal end 126 and/or second distal end 138 to facilitate positioning of first leadlet 120 and/or second leadlet 134, respectively.

Elongate member 154 may include a tip 155 configured to penetrate tissue of heart 102 to form a puncture sized to allow first leadlet 120 and/or second leadlet 134 to be inserted therethrough. In some examples, first leadlet 120 and/or second leadlet 134 may be configured to allow elongate member 154 to extend beyond first distal end 126 and/or second distal end 138, respectively. For example, first bearing structure 156 may define a first bearing structure opening 164 through which elongate member 154 may extend beyond first distal end 126. Similarly, second bearing structure 158 may define a second bearing structure opening 165 at second distal end 138 through which elongate member 154 may extend beyond second distal end 138.

Housing 110 may mechanically support circuitry 130. Circuitry 130 may be one or more of sensing circuitry (e.g., for sensing cardiac signals), therapy delivery circuitry (e.g., for generating cardiac pacing pulses), or processing circuitry for controlling the functionality of IMD 108. Circuitry 130 may be electrically coupled to first electrode 128, second electrode 140, and third electrode 142. Circuitry 130 may be configured to deliver CRT to heart 102 using first electrode 128, second electrode 140, third electrode 142, and return electrode 152 carried by housing 110. For example, first electrode 128 carried by first leadlet 120 may activate the left ventricular lateral wall; second electrode 140 carried by second leadlet 134 may activate the ventricular septum; and third electrode 142 may activate the atrium. Circuitry 130 may be configured to deliver VfA therapy and/or LBB therapy to heart 102 using second electrode 140 and return electrode 152. Circuitry 130 may be configured to deliver therapy to heart 102 using third electrode 142 and return electrode 152.

Referring now to FIG. 2B, IMD 108 may include fixation device 118 configured to engage tissue. That is, fixation device 118 may be configured to substantially maintain contact between IMD distal end 114 and tissue at a target implant site (e.g., target implant site 104 (FIG. 1)). In examples, and as shown in FIG. 2B, fixation device 118 may include one or more fixation tines attached and spaced from one another around a perimeter of IMD distal end 114, such as tine 166A, tine 166B, and/or tine 166C (collectively, “tines 166”), configured to engage tissue. However, other examples of fixation device 118 are contemplated by this disclosure, including fixation devices with a different number of tines (e.g., one, four, eight, etc.), a different arrangement of tines (e.g., about longitudinal axis 150 of IMD 108), different types of tines (e.g., helically-shaped fixation tines), and/or the like.

Any of tines 166 may be a deep tine, a shallow tine, and/or the like. Any of tines 166 may have elastic or superelastic properties, and may, in some cases, be configured to pierce and potentially penetrate into or through the tissue. In examples, one or more of tines 166 are configured to function as a distal electrode. Any of tines 166 may be configured to pierce through or penetrate the entire thickness of the tissue, potentially reaching a heart chamber opposite the septum from the chamber of implantation of IMD 108. In some examples, any of tines 166 may be configured to pierce the septum partially without reaching the chamber opposite the septum from the chamber of implantation of IMD 108.

In examples, tines 166 include a fixed end 168A, a fixed end 168B, and a fixed end 168C (collectively, “fixed ends 168”) coupled to IMD distal end 114 and a free end 170A, a free end 170B, and a free end 170C (collectively, “free ends 170”) opposite fixed ends 168. In examples, free ends 170 are configured to penetrate tissue. Tines 166 may be biased such that at least some portion of tines 166 expands radially as tines 166 pass through a distal opening defined by a distal end of delivery catheter 106. For example, tines 166 may be biased to drive free ends 170 radially outward from longitudinal axis 150 as free ends 170 pass through the distal opening of delivery catheter 106. The biasing tending to drive free ends 170 radially outward as tines 166 extend through the distal opening of delivery catheter 106 may cause tines 166 to substantially grasp tissue and more securely anchor IMD 108 within an atrium of heart 102. Free ends 170 may pierce the tissue and may act to pull IMD 108 and other portions of medical system 100 toward implant site 104 as tines 166 elastically bends or curves radially outward.

The biasing of tines 166 tending to drive free ends 170 radially outward may cause tines 166 to assume any general shape. In some examples, the biasing of tines 166 tends to cause tines 166 to position such that free ends 170 establishes a position proximal to a midpoint 167A, a midpoint 167B, and a midpoint 167C (collectively, “midpoints 167”) between respective fixed ends 168 and free ends 170. In some examples, the biasing of tines 166 tends to cause tines 166 to position such that free ends 170 remains distal to midpoints 167 between fixed ends 168 and free ends 170.

Fixation device 118 may include a conductor, such as an electrically conductive material, having a non-conductive coating, such as polytetrafluoroethylene (PTFE), a portion (e.g., a distal end of fixation device 118) of the conductive material being exposed to the tissue in which fixation device 118 is embedded upon implantation of IMD 108. Circuitry 130 of IMD 108 may be configured to generate and deliver electrical pulse therapy to the tissue proximate to fixation device 118 via an electrode formed by a portion of fixation device 118, through the tissue, to return electrode 152. In some examples, at least some portion of fixation device 118 is electrically active, in which case it may deliver stimulation therapy in addition to or alternative to stimulation therapy delivered by second electrode 140 and/or third electrode 142. Fixation device 118 may include one or more sections, such as an elastically deformable material preset into one or more curved sections and one or more optional substantially straight sections. Fixation device 118 may be formed to have a preset shape and may be superelastic, e.g., made of the nickel-titanium alloy Nitinol. In examples, fixation device 118 defines a ribbon shape configured to deform along a plane normal to longitudinal axis 150 and resist twisting outside of the plane. In some examples, fixation device 118 may include two or more curved sections (e.g., knuckles).

Fixation device 118 may be configured to have a target deflection stiffness and a target deployment stiffness. The target deflection stiffness may include a measure of a resistance to force applied to IMD 108 in a proximal direction when fixation device 118 is engaged with tissue at target implant site 104. In some examples, the target deflection stiffness may be selected to enable fixation device 118 to deflect a predetermined amount to enable visualization of fixation device 118 under fluoroscopy. In examples, the target deflection stiffness may be within a range from about 0.2 N to about 0.8 N, such as about 0.3 N to about 0.6 N. The deployment stiffness may include a measure of a force applied by fixation device 118 as fixation device 118 moves from a deformed configuration to an undeformed configuration upon deployment of IMD 108 from the distal opening of delivery catheter 106 such that the distal end of fixation device 118 penetrates the atrial or ventricular myocardium. In examples, the target deployment stiffness may be within a range from about 0.6 N to about 1.2 N.

In examples, IMD 108 includes a retrieval structure 172 fixedly attached to or formed integrally with IMD proximal end 112. Retrieval structure 172 may be configured for temporarily tethering IMD 108 to delivery catheter 106 or another delivery catheter or retrieval catheter. Retrieval structure 172 may be configured to couple to tether assemblies. A tether assembly may include a tether head assembly, a tether handle assembly, and a pull wire. The tether head assembly may be attached to the pull wire and configured to releasably retain an attachment member of a medical device (e.g., an intracardiac device). In examples, a tether handle assembly is configured to retain the pull wire attached to the tether head assembly.

IMD 108 may include a marker 174, which in some examples may be a radiopaque marker. Marker 174 may be visible via medical imaging such as fluoroscopy and allow a clinician to view and adjust the rotational orientation of IMD 108 to achieve a desired trajectory and/or a desired advancement path of fixation device 118 to target implant site 104.

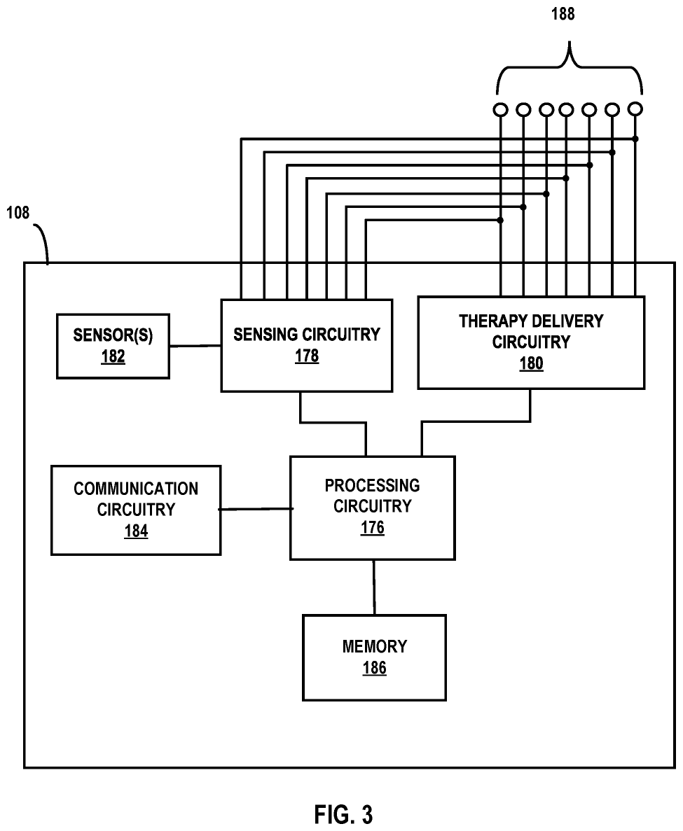

FIG. 3 is a functional block diagram illustrating an example configuration of IMD 108. As shown in FIG. 3, IMD 108 includes processing circuitry 176, sensing circuitry 178, therapy delivery circuitry 180, sensors 182, communication circuitry 184, and memory 186. Processing circuitry 176, sensing circuitry 178, therapy delivery circuitry 180, and/or communication circuitry 184 may be examples of circuitry 130, In some examples, memory 186 includes computer-readable instructions that, when executed by processing circuitry 176, cause IMD 108 and processing circuitry 176 to perform various functions attributed to IMD 108 and processing circuitry 176 herein. Memory 186 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 176 may include fixed function circuitry and/or programmable processing circuitry. Processing circuitry 176 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 176 may include multiple components, such as any combination of one or more microprocessors, one or more controllers, one or more DSPs, one or more ASICs, or one or more FPGAs, as well as other discrete or integrated logic circuitry. The functions attributed to processing circuitry 176 herein may be embodied as software, firmware, hardware or any combination thereof.

In some examples, processing circuitry 176 may receive (e.g., from an external device), via communication circuitry 184, a respective value for each of a plurality of cardiac sensing parameters, cardiac therapy parameters (e.g., cardiac pacing parameters), and/or electrode vectors. Processing circuitry 176 may store such parameters and/or electrode vectors in memory 186.

Therapy delivery circuitry 180 and sensing circuitry 178 are electrically coupled to electrodes 188, which may correspond to first electrode 128, second electrode 140, third electrode 142, return electrode 152, and/or other electrodes of medical system 100. Processing circuitry 176 is configured to control therapy delivery circuitry 180 to generate and deliver electrical therapy to heart 102 via electrodes 188. Electrical therapy may include, for example, pacing pulses, or any other suitable electrical stimulation. Processing circuitry 176 may control therapy delivery circuitry 180 to deliver electrical stimulation therapy via electrodes 188 according to one or more therapy parameter values, which may be stored in memory 186. Therapy delivery circuitry 180 may include capacitors, current sources, and/or regulators, in some examples.

In general, electrodes (e.g., first electrode 128, second electrode 140, etc.) of IMD 108 may be ring electrodes, multi-polar electrodes (e.g., segmented electrodes), or any other type of electrode configured to sense electrical signals from and deliver therapy signals to heart 102. In examples, first electrode 128 is a multi-polar electrode carried by first leadlet 120 near first distal end 126, second electrode 140 a multi-polar electrode carried by second leadlet 134 near second distal end 138, and third electrode 142 an atrial electrode carried by third leadlet 144 near third distal end 148. In some cases, one or more of electrodes 188, such as second electrode 140 and/or third electrode 142, may be carried by housing 110 (e.g., disposed on IMD distal end 114). In general, first electrode 128 may be disposed anywhere along a length of first leadlet 120, and in cases where second electrode 140 is carried by second leadlet 134, second electrode 140 may be disposed anywhere along a length of second leadlet 134. Each of electrode 188 may be configured to provide independent pacing, which may facilitate correctly placing electrodes 188 and/or obtaining a better electrical signal (lower threshold, lower impedance, etc.). The spacing between electrodes 188 may be variable.

In addition, processing circuitry 176 is configured to control sensing circuitry 178 to monitor signals from electrodes 188 in order to monitor electrical activity of heart 102. Sensing circuitry 178 may include circuits that acquire electrical signals, such as filters, amplifiers, and analog-to-digital circuitry. Electrical signals acquired by sensing circuitry 178 may include intrinsic and/or paced cardiac electrical activity, such as atrial depolarizations and/or ventricular depolarizations. Sensing circuitry 178 may filter, amplify, and digitize the acquired electrical signals to generate raw digital data. Processing circuitry 176 may receive the digitized data generated by sensing circuitry 178. In some examples, processing circuitry 176 may perform various digital signal processing operations on the raw data, such as digital filtering. In some examples, in addition to sensing circuitry 178, IMD 108 optionally may include sensors 182, which may be one or more pressure sensors and/or one or more accelerometers, as examples. Communication circuitry 184 may include any suitable hardware (e.g., an antenna), firmware, software, or any combination thereof for communicating with another device, e.g., external to the patient.

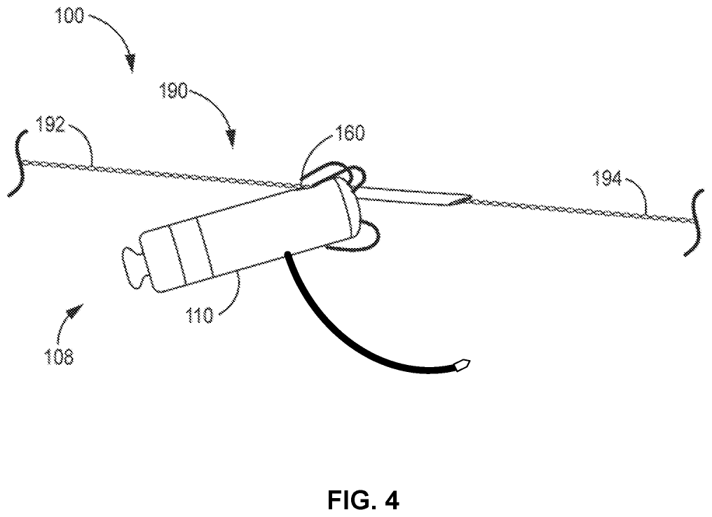

FIG. 4 is a conceptual diagram illustrating medical system 100 of FIG. 1, including implantable medical device 108 and a guidewire 190. Guidewire 190 may be configured to transition between and unexpanded state and an expanded state in response to, for example, actuation of a user device. In examples, guidewire 190 has a proximal portion 192 and a distal portion 194. Distal portion 194 may include a distal end (not shown in FIG. 4) configured to penetrate a septal boundary of heart 102. Proximal portion 192 may include a proximal end (not shown in FIG. 4) that in turn includes a user device (e.g., a trigger) configured to cause distal portion 194 to expand when the septal boundary separates proximal portion 192 and distal portion 194. Inner lumen 160 may be configured to receive guidewire 190 such that IMD 108 may translate over guidewire 190 toward a target implant site 104. IMD 108 may be delivered to the tissue by translating over guidewire 190. In some examples, guidewire 190 is made from nitinol.

For example, a clinician may place guidewire 190 across the septum into a left ventricle (LV) chamber near or through the LBB. IMD 108 may then be translated over the guidewire until IMD 108 is delivered to target implant site 104. When the septal boundary separates proximal portion 192 and distal portion 194 of guidewire 190, the clinician may use the user device to cause distal portion 194 to expand, resisting movement of guidewire 190 in the proximal direction. Guidewire 190 may include a tip configured to penetrate the tissue to form a puncture through which leadlet 114 may be inserted.

In examples, guidewire 190 may be removed from target implant site 104 after delivery and implantation of IMD 108. In other examples, guidewire 190 is configured to be removed before IMD 108 is implanted into the tissue. For example, guidewire 190 may be removed before IMD 108 reaches the LV endocardium, reducing the probability that IMD 108 exceeds a desired penetration depth of the tissue.

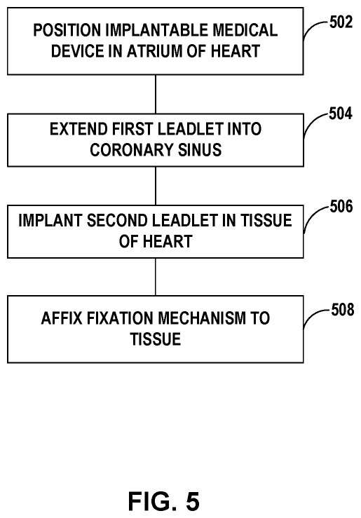

FIG. 5 is an example technique for positioning medical system 100 within heart 102. IMD 108 may be delivered to implant site 104 in heart 102 (502). In some examples, the physician may insert IMD 108 into catheter lumen of delivery catheter 106 and implant IMD 108 at implant site 104 by exerting a distal force on IMD 108. Implant site 104 may include an appendage or triangle of Koch region of the RA, or some other portion of heart 102, or other locations within a body of a patient. Other pathways or techniques may be used to guide delivery catheter 106 into other implant sites within the body of the patient.

First leadlet 120 may extend within coronary sinus 122 of heart 102 (504). For example, first leadlet body 132 may be positioned within coronary sinus 122 when fixation device 118 secures IMD distal end 114 to heart tissue (e.g., in the atrium). First leadlet 120 may carry (e.g., mechanically support) a first electrode 128. First electrode 128 may deliver therapy to heart 102. First electrode 128 may be a multi-polar electrode (including two electrodes, four electrodes, etc.) configured to deliver LV therapy in a substantially non-invasive manner when at least a portion of first leadlet 120 is positioned within coronary sinus 122. In some examples, first electrode 128 may be a penetrating electrode configured to penetrate tissue of coronary sinus 122.

First leadlet 120 may receive elongate member 154, such as a stylet, configured to exert or cause first leadlet 120 to exert a distal force on first distal end 126. In some examples, first leadlet 120 may define first bearing structure 156 (e.g., a lumen, a casing, etc.) configured to transmit a distal force to first distal end 126 when elongate member 154 exerts the distal force on first bearing structure 156. For example, first bearing structure 156 may be a casing extending from an outer surface of first leadlet 120 configured to receive a distal force. First bearing structure 156 may receive elongate member 154, and elongate member 154 may mechanically communicate with first bearing structure 156, in this way exerting a distal force on first bearing structure 156 and, thus, first distal end 126.

Second leadlet 134 may implant into tissue of heart 102 (506). Second distal end 138 of second leadlet 134 may be positioned within heart tissue when fixation device 118 secures IMD distal end 114 to heart tissue (e.g., in the atrium). In some examples, second distal end 126 may penetrate tissue of heart 102 when a distal force is applied to second leadlet 134 (e.g., via IMD 108) to form a puncture sized to allow second electrode 140 to be inserted therethrough. Second electrode 140 may be a multi-polar electrode. Second electrode 140 may deliver VfA therapy when second distal end 138 is implanted in a right atrium of heart 102. Additionally or alternatively, second electrode 140 may deliver LBB therapy when second distal end 138 is implanted in a right ventricle septum of heart 102.

Second leadlet 134 may insert or cause insertion of second electrode 140 into heart tissue. In some examples, second leadlet 134 may receive elongate member 154 configured to exert a distal force on second distal end 138. In some examples, second leadlet 134 may define second bearing structure 158 (e.g., a lumen, a casing, etc.) configured to transmit a distal force to second distal end 138 when elongate member 154 exerts the distal force on second bearing structure 158. Second bearing structure 158 may be a lumen with a surface configured to receive a distal force. Second bearing structure 158 may receive elongate member 154, and elongate member 154 may mechanically communicate with second bearing structure 158, in this way exerting a distal force on second bearing structure 158 and, thus, second distal end 138.

In some examples, IMD 108 may carry third electrode 142 that contacts a surface of heart tissue when fixation device 118 secures IMD distal end 114 to heart tissue in the atrium. Third electrode 142 may be attached to IMD distal end 114, housing wall 116, or another portion of IMD 108. Alternatively, third leadlet 144 may mechanically support third electrode 142, for example, near third distal end 148. Third electrode 142 may deliver therapy to heart 102. In examples, third electrode 142 is an atrial electrode.

IMD 108 may include first electrode 128, second electrode 140, and third electrode 142 in various combinations. For example, IMD 108 may include first leadlet 120 carrying first electrode 128. In another example, IMD 108 may include first leadlet 120 carrying first electrode 128 and second leadlet 134 carrying second electrode 140. In yet another example IMD 108 may include first leadlet 120 carrying first electrode 128 and third leadlet 144 carrying third electrode 142. In yet another example, IMD 108 may include first leadlet 120 carrying first electrode 128, second leadlet 134 carrying second electrode 140, and third leadlet 144 carrying third electrode 142.

Fixation device 118 of IMD 108 may engage tissue of heart 102 (508). In some examples, elongate member 154 may apply a distal force to leadlet 114 until fixation device 118 penetrates tissues of heart 102. A clinician may then confirm adequate fixation (e.g., that IMD 108 is secured well enough to maintain an orientation of IMD 108 with respect to implant site 104 to prevent motion of IMD 108 in a proximal direction) of fixation device 118. For example, a pull test or a tug test may be performed under fluoroscopy to confirm that fixation device 118 has engaged the tissue to confirm adequacy of implantation of IMD 108. The pull test or tug test may include the clinician pulling or tugging on IMD 108, e.g., via a tether coupled to a proximal end of IMD 108, and observing movement of fixation device 118 to determine if fixation device 118 is engaged in tissue. For example, fixation device 118 that is embedded in tissue may deflect or bend as IMD 108 is pulled or tugged in the proximal direction.

The disclosure includes the following examples.

Example 1: A medical system includes an implantable medical device configured to be positioned within an atrium of a heart and including a housing carrying a return electrode, wherein the housing includes a proximal end and a distal end; a first leadlet including a first proximal end and a first distal end, wherein the first proximal end is attached to the implantable medical device, and wherein the first leadlet carries a first electrode; and a fixation device attached to the distal end of the housing, wherein the first leadlet is configured to extend within a coronary sinus of the heart when the fixation device secures the distal end to heart tissue in the atrium.

Example 2: The medical system of example 1, wherein the first electrode is a multi-polar electrode configured to deliver left ventricular therapy when at least a portion of the first leadlet is positioned within the coronary sinus.

Example 3: The medical system of example 1 or 2, wherein the implantable medical device further includes therapy delivery circuitry mechanically supported within the housing, wherein the first electrode and the return electrode are electrically coupled to the therapy delivery circuitry, and wherein the therapy delivery circuitry is configured to deliver cardiac resynchronization therapy to the heart using the first electrode and the return electrode.

Example 4: The medical system of any of examples 1 through 3, wherein the first distal end is configured to penetrate coronary sinus tissue when a distal force is applied to the first leadlet to form a puncture sized to allow the first electrode to be inserted therethrough.

Example 5: The medical system of example 4, wherein the first leadlet is configured to insert the first electrode into the coronary sinus tissue.

Example 6: The medical system of any of examples 1 through 5, wherein the first leadlet carries the first electrode on a first leadlet body between first proximal end and the first distal end, and wherein the first leadlet is configured to position the first leadlet body within the coronary sinus when the fixation device secures the distal end to heart tissue in the atrium.

Example 7: The medical system of any of examples 1 through 6, wherein the first proximal end is attached to the distal end of the housing.

Example 8: The medical system of any of examples 1 through 7, wherein the housing includes a housing wall extending between the proximal end of the housing and the distal end of the housing, wherein the first proximal end is attached to the housing wall.

Example 9: The medical system of any of examples 1 through 8, wherein the first proximal end is attached to the proximal end of the housing.

Example 10: The medical system of any of examples 1 through 9, wherein the first distal end includes a fixation structure configured to resist motion of the first electrode in a proximal direction when the first distal end is implanted in the heart tissue.

Example 11: The medical system of any of examples 1 through 10, wherein the first distal end includes a fixation structure configured to resist motion of the first electrode in a proximal direction when the first distal end is implanted in the heart tissue.

Example 12: The medical system of any of examples 1 through 11, wherein the first leadlet is configured to receive an elongate member, and wherein the first distal end is configured to move in a distal direction when the first leadlet receives the elongate member and a distal force is exerted on the elongate member.

Example 13: The medical system of example 12, wherein the elongate member is a stylet.

Example 14: The medical system of example 12 or 13, wherein the elongate member includes a tip configured to penetrate the heart tissue to form a puncture sized to allow the first leadlet to be inserted therethrough, and wherein the first leadlet is configured to allow the elongate member to extend beyond the first distal end.

Example 15: The medical system of any of examples 12 through 14, wherein the implantable medical device defines an inner lumen extending from a lumen opening defined by the implantable medical device toward the first distal end, wherein the inner lumen is configured to receive the elongate member.

Example 16: The medical system of any of examples 12 through 15, wherein the first leadlet defines a first bearing structure configured to transmit a distal force to the first distal end when the elongate member exerts a distal force on the first bearing structure.

Example 17: The medical system of example 16, wherein the first bearing structure is an inner lumen defined by the first leadlet.

Example 18: The medical system of example 16, wherein the first bearing structure is a casing extending from an outer surface of the first leadlet.

Example 19: The medical system of any of examples 1 through 18, further including a second leadlet including a second proximal end and a second distal end, wherein the second leadlet carries a second electrode, wherein the second proximal end is attached to the implantable medical device, and wherein the second distal end is configured to penetrate the heart tissue when a distal force is applied to the second leadlet to form a puncture sized to allow the second electrode to be inserted therethrough.

Example 20: The medical system of example 19, wherein the second distal end is configured to position within the heart tissue when the fixation device secures the distal end of the housing to the heart tissue in the atrium.

Example 21: The medical system of example 19 or 20, wherein the second electrode is a multi-polar electrode configured to deliver ventricle-from-atrium cardiac therapy when the second distal end is implanted in a ventricular tissue of the heart.

Example 22: The medical system of any of examples 19 through 21, wherein the second electrode is a multi-polar electrode configured to deliver left bundle branch pacing therapy when the second distal end is implanted in a right ventricle septum of the heart.

Example 23: The medical system of any of examples 19 through 22, wherein the implantable medical device further includes therapy delivery circuitry mechanically supported within the housing, wherein the second electrode and the return electrode are electrically coupled to the therapy delivery circuitry, and wherein the therapy delivery circuitry is configured to deliver ventricle-from-atrium cardiac therapy to the heart using the second electrode and the return electrode when the second distal end is implanted in the heart tissue.

Example 24: The medical system of any of examples 19 through 23, wherein the second proximal end is attached to a distal end of the housing.

Example 25: The medical system of any of examples 19 through 24, wherein the distal end of the second leadlet includes a fixation structure configured to resist motion of the second electrode in a proximal direction when the second distal end is implanted in the heart tissue.

Example 26: The medical system of any of examples 19 through 25, wherein the second leadlet is configured to receive an elongate member, and wherein the second distal end is configured to move in a distal direction when the second leadlet receives the elongate member and a distal force is exerted on the elongate member.

Example 27: The medical system of example 26, wherein the elongate member is a stylet.

Example 28: The medical system of example 26 or 27, wherein the elongate member includes a tip configured to penetrate the heart tissue to form a puncture sized to allow the second leadlet to be inserted therethrough, and wherein the second leadlet is configured to allow the elongate member to extend beyond the second distal end of the second leadlet.

Example 29: The medical system of any of examples 26 through 28, wherein the implantable medical device defines an inner lumen extending from a lumen opening defined by the implantable medical device toward the second distal end of the second leadlet, wherein the inner lumen is configured to receive the elongate member.

Example 30: The medical system of any of examples 26 through 29, wherein the second leadlet defines a second bearing structure configured to transmit a distal force to the second distal end when the elongate member exerts a distal force on the second bearing structure.

Example 31: The medical system of example 30, wherein the second bearing structure is an inner lumen defined by the second leadlet.

Example 32: The medical system of example 30, wherein the second bearing structure is a casing extending from an outer surface of the second leadlet.

Example 33: The medical system of any of examples 1 through 32, further including a third electrode configured to contact a surface of the heart tissue when the fixation device secures the implantable medical device to the heart tissue.

Example 34: The medical device of example 33, wherein the distal end of the housing mechanically supports the third electrode.

Example 35: The medical system of example 33 or 34, further including a third leadlet including a third proximal end and a third distal end, wherein the third proximal end is attached to the implantable medical device, and wherein the third leadlet carries the third electrode.

Example 36: The medical system of any of examples 33 through 35, wherein the implantable medical device further includes therapy delivery circuitry mechanically supported within the housing, wherein the third electrode and the return electrode are electrically coupled to the therapy delivery circuitry, and wherein the therapy delivery circuitry is configured to provide stimulation to the heart using the third electrode and the return electrode.

Example 37: The medical system of any of examples 33 through 36, wherein the third proximal end is attached to the distal end of the housing.

Example 38: The medical system of any of examples 33 through 37, wherein the housing includes a housing wall extending between the proximal end of the housing and the distal end of the housing, wherein the third proximal end is attached to the housing wall.

Example 39: The medical system of example 38, wherein the implantable medical device defines an inner lumen extending from a lumen opening defined by the housing wall towards one or more of the first distal end or the second distal end, wherein the inner lumen is configured to enable the implantable medical device to translate over a guidewire when the guidewire extends through the inner lumen.

Example 40: The medical system of example 39, further including the guidewire, wherein the guidewire includes a proximal portion and a distal portion, wherein the distal portion includes a distal end configured to penetrate a septal boundary, and wherein the proximal portion includes a user device configured to cause the distal portion to expand when the septal boundary separates the proximal portion and the distal portion.

Example 41: The medical system of any of examples 1 through 40, wherein the fixation device includes one or more tines, wherein an individual tine in the one or more tines includes a fixed end attached to the distal end of the housing and a free end opposite the fixed end, and wherein the individual tine is resiliently biased to pivot the free end radially outward from the longitudinal axis.

Example 42: The medical system of any of examples 1 through 41, wherein the fixation device is configured to resist motion of the implantable medical device in a proximal direction when the fixation device affixes the implantable medical device to the heart tissue.

Example 43: The medical system of any of examples 1 through 42, further including a delivery catheter defining a catheter lumen, wherein the delivery catheter defines a lumen opening to the catheter lumen at a distal end of the delivery catheter, and wherein the catheter lumen and the lumen opening are sized to allow the medical device to translate through the catheter lumen and through the lumen opening.

Example 44: A method of implanting a medical system includes positioning an implantable medical device within an atrium of a heart, wherein the implantable medical device includes a housing carrying a return electrode, wherein the housing includes a proximal end and a distal end; securing a fixation device, attached to the distal end of the housing, to the heart tissue; and extending a first leadlet into a coronary sinus of the heart, wherein the first leadlet includes a first proximal end and a first distal end, wherein the first proximal end is attached to the implantable medical device, and wherein the first leadlet carries a first electrode.

Example 45: The method of example 44, wherein the medical system includes the medical system of any of examples 1 through 44.

Example 46: The method of example 44 or 45, further including delivering ventricle-from-atrium cardiac therapy (“VfA therapy”) via the implanted medical system.

Example 47: The method of example 44 or 45, further including delivering left bundle branch pacing therapy (“LBB therapy”) via the implanted medical system.

Example 48: The method of example 44 or 45, further including delivering cardiac resynchronization therapy (“CRT”) via the implanted medical system.

Example 49: A medical system includes an implantable medical device configured to be positioned within an atrium of a heart and including a housing carrying a return electrode, wherein the housing includes a proximal end and a distal end; a first leadlet including a first proximal end and a first distal end, wherein the first proximal end is attached to the implantable medical device, and wherein the first leadlet carries a first electrode; a second leadlet including a second proximal end and a second distal end, wherein the second leadlet carries a second electrode, wherein the second proximal end is attached to the implantable medical device, and wherein the second distal end is configured to penetrate the heart tissue when a distal force is applied to the second leadlet to form a puncture sized to allow the second electrode to be inserted therethrough; a third electrode disposed on the distal end of the housing; and a fixation device attached to the distal end of the housing, wherein the first leadlet is configured to extend within a coronary sinus of the heart when the fixation device secures the distal end to heart tissue in the atrium, and wherein the third electrode is configured to contact a surface of the heart tissue when the fixation device secures the implantable medical device to the heart tissue.

Example 50: The medical system of example 49, wherein the implantable medical device further includes therapy delivery circuitry mechanically supported within the housing, wherein the first electrode, the second electrode, the third electrode, and the return electrode are electrically coupled to the therapy delivery circuitry, and wherein the therapy delivery circuitry is configured to deliver cardiac resynchronization therapy to the heart using the first electrode, the second electrode, the third electrode, and the return electrode.

Example 51: The medical system of example 49 or 50, wherein the medical system is configured to deliver ventricle-from-atrium cardiac therapy (“VfA therapy”).

Example 52: The medical system of any of examples 49 through 51, wherein the medical system is configured to deliver left bundle branch pacing therapy (“LBB therapy”).

Example 53: The medical system of any of examples 49 through 52, wherein the medical system is configured to deliver cardiac resynchronization therapy (“CRT”).

Various examples of the disclosure have been described. Any combination of the described systems, operations, or functions is contemplated. These and other examples are within the scope of the following claims.

What is claimed is:

1. A medical system comprising:
an implantable medical device configured to be positioned within an atrium of a heart and comprising a housing carrying a return electrode, wherein the housing comprises a proximal end and a distal end;
a first leadlet comprising a first proximal end and a first distal end, wherein the first proximal end is attached to the implantable medical device, and wherein the first leadlet carries a first electrode;
a second leadlet comprising a second proximal end and a second distal end, wherein the second proximal end is attached to the implantable medical device, and wherein the second leadlet carries a second electrode; and
a fixation device attached to the distal end of the housing, wherein the first leadlet is configured to extend within a coronary sinus of the heart when the fixation device secures the distal end to heart tissue in the atrium, and wherein the second distal end is configured to engage the heart tissue when the fixation device secures the distal end of the housing to the heart tissue in the atrium.

2. The medical system of claim 1, wherein the first leadlet is configured to position the first electrode within the coronary sinus when the first leadlet extends within the coronary sinus.

3. The medical system of claim 1,
wherein the implantable medical device further comprises therapy delivery circuitry mechanically supported within the housing,
wherein the first electrode, the second electrode, and the return electrode are electrically coupled to the therapy delivery circuitry, and
wherein the therapy delivery circuitry is configured to:
deliver therapy to the heart using the first electrode and the return electrode when the first leadlet extends within the coronary sinus; and
deliver therapy to the heart using the second electrode and the return electrode when the second distal end engages the heart tissue.

4. The medical system of claim 1, wherein the second distal end is configured to penetrate the heart tissue when a distal force is applied to the second leadlet.

5. The medical system of claim 1, wherein the first distal end is configured to penetrate coronary sinus tissue when a distal force is applied to the first leadlet.

6. The medical system of claim 5, wherein the first leadlet is configured to position the first electrode in the coronary sinus tissue when the first distal end penetrates the coronary sinus tissue.

7. The medical system of claim 5, wherein the first leadlet is configured to receive an elongate member, and wherein the elongate member is configured to apply the distal force to the first leadlet when the first leadlet receives the elongate member and a distal force is exerted on the elongate member.

8. The medical system of claim 7, wherein the implantable medical device defines an inner lumen extending from a lumen opening defined by the implantable medical device toward the first distal end, and wherein the inner lumen is configured to receive the elongate member.

9. The medical system of claim 7, wherein the first leadlet defines a first bearing structure configured to transmit the distal force to the first distal end when the elongate member exerts the distal force on the first bearing structure.

10. The medical system of claim 5, wherein the first distal end is configured to move in a distal direction to penetrate the coronary sinus tissue, and wherein the first distal end comprises a fixation structure configured to resist motion in a proximal direction when the first distal end penetrates the coronary sinus tissue.

11. The medical system of claim 1, further comprising the elongate member, wherein the elongate member comprises a tip configured to penetrate the heart tissue to form a puncture sized to allow the second leadlet to be inserted therethrough, and wherein the second leadlet is configured to allow the elongate member to extend beyond the second distal end of the second leadlet.

12. The medical system of claim 7, wherein the implantable medical device defines an inner lumen extending from a lumen opening defined by the implantable medical device toward the second distal end of the second leadlet, wherein the inner lumen is configured to receive the elongate member.

13. The medical system of claim 1, further comprising a third electrode configured to contact a surface of the heart tissue when the fixation device secures the implantable medical device to the heart tissue.

14. The medical system of claim 13,
wherein the third electrode is electrically coupled to the therapy delivery circuitry, and
wherein the therapy delivery circuitry is configured to provide stimulation to the heart using the third electrode.

15. The medical system of claim 13, further comprising a third leadlet comprising a third proximal end and a third distal end, wherein the third proximal end is attached to the implantable medical device, and wherein the third leadlet carries the third electrode.

16. A medical system comprising:
an implantable medical device configured to be positioned within an atrium of a heart and comprising a housing carrying a return electrode, wherein the housing comprises a proximal end and a distal end;
a first leadlet comprising a first proximal end and a first distal end, wherein the first proximal end is attached to the implantable medical device, and wherein the first leadlet carries a first electrode;
a second leadlet comprising a second proximal end and a second distal end, wherein the second proximal end is attached to the implantable medical device, wherein the second leadlet carries a second electrode, and wherein the second distal end is configured to penetrate the heart tissue when a distal force is applied to the second leadlet;
a third electrode disposed on the distal end of the housing; and
a fixation device attached to the distal end of the housing,
wherein the first leadlet is configured to extend within a coronary sinus of the heart when the fixation device secures the distal end to heart tissue in the atrium,
wherein the first leadlet is configured to position the first electrode within the coronary sinus when the first leadlet extends within the coronary sinus, and
wherein the third electrode is configured to contact a surface of the heart tissue when the fixation device secures the implantable medical device to the heart tissue.

17. The medical system of claim 16, further comprising therapy delivery circuitry configured to at least one of:
deliver therapy to the heart using the first electrode when the first leadlet extends within the coronary sinus,
deliver therapy to the heart using the second electrode when the second distal end penetrates the heart tissue, or
deliver therapy to the heart using the third electrode when the third electrode contacts the surface of the heart tissue.

18. The medical system of claim 17, wherein the therapy delivery circuitry is configured to deliver one or more of:
ventricle-from-atrium cardiac therapy (“VfA therapy”) or left bundle branch pacing therapy (“LBB therapy”) using the first electrode, or
cardiac resynchronization therapy (“CRT”) using the second electrode.

19. A method of implanting a medical system, the method comprising:
positioning an implantable medical device within an atrium of a heart, wherein the implantable medical device comprises:
a housing carrying a return electrode, wherein the housing comprises a proximal end and a distal end;
a first leadlet comprising a first proximal end and a first distal end, wherein the first proximal end is attached to the implantable medical device, and wherein the first leadlet carries a first electrode;
a second leadlet comprising a second proximal end and a second distal end,
wherein the second proximal end is attached to the implantable medical device, and
wherein the second leadlet carries a second electrode; and
a fixation device attached to the distal end of the housing;
securing the fixation device to the heart tissue in the atrium;
extending the first leadlet of the implantable medical device into a coronary sinus of the heart; and
positioning the second distal end within the heart tissue.

20. The method of claim 18, further comprising delivering, using therapy delivery circuitry, at least one of:
therapy to the heart using the first electrode when the first leadlet extends within the coronary sinus; or
therapy to the heart using the second electrode when the second distal end positions within the heart tissue.
